(19) United States
(12) Patent Application Publication
(10) Pub. No.: US 2011/0288017 A1
(43) Pub. Date: Nov. 24, 2011

(54) APO-LIPOPROTEIN PROPEPTIDE

(76) Inventor: Leslie Frost, Ona, WV (US)

(21) Appl. No.: 13/038,196

(22) Filed: Mar. 1, 2011

Related U.S. Application Data
(60) Provisional application No. 61/339,086, filed on Mar. 1, 2010.

(51) Int. Cl. A61K 38/08; C07H 21/04; A61P 3/06; A61P 7/00; A61P 25/28; A61P 9/10; A61P 9/08; A61P 37/00; A61P 29/00; C07K 7/06; A61P 9/00

(52) U.S. Cl. 514/7.4; 530/329; 536/23.5

(57) ABSTRACT

This invention relates generally to a peptide being a fragment of the mammalian apolipoprotein AI peptide, said peptide comprising the sequence RHFWQQ (human, macaque and pig), the sequence WEFWQQ (rat), and the sequence WHVWQQ (mouse), or suitable modifications thereof, or equivalent synthetic peptides. In contrast to the entire protein, these short peptides have the ability to enter the nucleus of a cell. By binding directly to the DNA or to a protein that is attached to the DNA, these peptides undertake a role as a transcription factor. In particular, these peptides bind to the LXRβ receptor within the nucleus of the human cell. Therefore, they may be used as a drug to effectively treat disorders such as hyperlipidemia, inflammation (arthritis), and Alzheimer's disease.

### CROSS REFERENCE TO RELATED APPLICATION

This application claims priority from U.S. Provisional Application 61/339,086, filed on Mar. 1, 2010. The teachings in the provisional application are incorporated herein by reference.

### FIELD OF THE INVENTION

The present invention relates generally to fragments of the mammalian apolipoprotein AI peptide, and their use in the treatment of dyslipoproteinemia (hypercholesterol, cardiovascular disease, atherosclerosis, restenosis, and septic shock), inflammation (arthritis), and Alzheimer's disease.

### BACKGROUND OF THE INVENTION

Cholesterol metabolism is very complex and involves blood plasma lipoproteins such as high density lipoproteins (“HDL”), low density lipoproteins (“LDL”), very low density lipoproteins (“VLDL”) and chylomicrons. Plasma lipoproteins are synthesized in the endoplasmic reticulum of the liver and the intestines. Their specific site of synthesis, half-life in the blood plasma, and their transport function vary.

Most of the components of chylomicrons are synthesized in the intestinal cells. The chlyomicrons transport triacylglycerol and cholesterol ester from the intestines to other tissues in the body. The half-life for clearance of chylomicrons from plasma is 4 to 5 minutes.

The components for HDL are synthesized in the liver and released to the plasma. The function of HDL is to carry cholesterol esters and fatty acids from the tissues back to the liver, and HDL can also transfer cholesterol esters to LDL. The half-life of HDL in the plasma is 5 to 6 days.

VLDL functions in the transport of lipid and cholesterol from the liver to other tissues. The half-life for VLDL is one to three hours. As VLDL is depleted of triacylglycerols, the particle is converted into LDL and enriched in cholesterol esters from HDL.

Cholesterol is either taken in through the diet or biosynthesized primarily in the liver. LDL carries cholesterol esters from the liver to other cells of the body, which includes the adrenals and gonads where cholesterol is converted to steroid hormones. The steroid hormones are eventually secreted as glycosyl conjugates in the urine. HDLs carry cholesterol esters from other cells to the liver. The enzyme lecithin-cholesterol acyltransferase (“LCAT”) is associated with HDL in plasma and is activated by apolipoprotein AI (“Apo AI”). This enzyme catalyzes the transfer of an acyl group from phosphatidylcholine on the surface of the HDL particle to the cholesterol to form the cholesterol ester. Another enzyme associated with the HDL particle is cholesterol ester transfer protein which catalyzes the transfer of cholesterol esters from HDL to VLDL or LDL. There are receptors for LDL on the surface of all cells. Once LDL binds to these receptors, the LDL particle is rapidly internalized by endocytosis. The LDL particles are degraded within the lysosomes. The cholesterol diffuses from the lysosomes and suppresses the activity of HMG-CoA reductase (the main regulatory enzyme in the biosynthesis of cholesterol), and stimulates the activity of acyl-CoA-cholesterol acyltransferase (“ACAT”). ACAT catalyzes the synthesis of cholesterol esters which are stored in the cell. The cholesterol also inhibits the synthesis of LDL receptors and limits the amounts of cholesterol taken into the cells.

The most important pathway for the degradation of cholesterol is the conversion of cholesterol to bile acids in the liver. In a normal adult, approximately 0.5 grams of cholesterol is converted to bile acids each day. The main regulation site for this conversion is the enzyme 7α-hydroxylase located in the endoplasmic reticulum. This enzyme is involved in the initial biosynthetic step of the pathway and results in the hydroxylation of the sterol at numerous, specific sites. The hydroxylation of cholesterol also requires NADPH-cytochrome P450 reductase. When cholesterol is transported back to the liver from other tissues, there should be a signal that this cholesterol is to be converted to bile acids for removal from the body, instead of recirculation back to the tissues.

Cardiovascular disease (“CVD”) is the general term for heart and blood vessels diseases, including atherosclerosis. Atherosclerosis is a chronic disease that causes a thickening of the innermost layer of large and medium arteries. It decreases blood flow and may therefore harm tissue in organs supplied by the affected blood vessel. Atherosclerosis is the major cause of CVD including myocardial infarction, stroke and peripheral artery disease. It is also a leading cause of death in the world. The disease is initiated by accumulation of lipoproteins, primarily LDL, in the extracellular matrix of the vessel. The LDL particles aggregate and are oxidized, thereby causing vascular injury. Atherosclerosis is a response to this injury that comprises inflammation and fibrosis. Therefore, there is an urgent need to develop safe and effective treatments for these diseases.

LDL and HDL are the major cholesterol carriers. LDL are believed to transport cholesterol from the liver to extrahepatic tissues in the body. On the other hand, HDL particles play a major role in the reverse process called reverse cholesterol transport (“RCT”)—i.e. return of excess cholesterol from peripheral tissues, including arterial lesion sites, to the liver for conversion to bile acids and excretion from the body. Thus, HDL act as scavengers of tissue cholesterol. Thus, high serum levels of HDL are considered as a negative risk factor.

In RCT, cellular HDL mediates cholesterol efflux by acting in conjunction with the cholesterol esterifying enzyme, LCAT. Cholesteryl ester accumulating in HDL can then follow a number of different fates: uptake in the liver in HDL containing apolipoprotein by LDL receptors, selective uptake of HDL cholesteryl ester in liver or other tissues involving scavenger receptor B1, or transfer to triglyceride-rich lipoproteins as a result of the activity of cholesteryl ester transfer protein, with subsequent uptake of triglyceride-rich lipoprotein remnants in the liver.

Many treatments for CVD involve lowering serum cholesterol and increasing HDL serum levels. Earlier drugs that lowered cholesterol could not safely elevate HDL levels and stimulate RCT.

It has been documented that high levels of Apo AI and HDL in serum have an inhibitory effect on the development of atherosclerosis (Lewis et al. Circ Res. 2005; 96: 1221-32; Rubin et al. Nature. 1991; 353: 265-7; Plump et al. Proc Natl Acad Sci USA. 1994; 91: 9607-11). The main lipid constituents of HDL complexes are various phospholipids, cholesterol (ester) and triglycerides. The most prominent apolipoprotein components are AI and AII, which determine the functional characteristics of HDL.

Apo AI is synthesized in the liver and the intestines, and it is the main constituent of HDL (64%) and chylomicrons. All lipoproteins contain a signal peptide at the N-terminus to direct these proteins to the endoplasmic reticulum (“ER”) for processing and secretion into the serum. The signal peptide is cleaved off in the ER. Apo AI is the only lipoprotein that contains an additional six amino acid propeptide on the N-terminus. This peptide is cleaved by an enzyme in the serum resulting in mature Apo AI (without the propeptide) and pro Apo AI (with the peptide still attached) circulating in the serum. The entire apolipoprotein with the propeptide still attached remains in the bloodstream, outside of cells, and functions to transport lipids and cholesterol through the bloodstream back to the kidneys (or it gives its cholesterol to LDL particles).

It is this biological function of Apo AI that has been the object of use in pharmaceutical compositions to treat CVD. However, Apo AI is a large protein that is difficult and expensive to produce. There are also issues related to storage, delivery, and half-life in vivo. Thus, shorter fragments of the protein are sought for their biologically active properties.

The mature Apo AI and pro Apo AI, and their modifications, are disclosed by Dasseux et al. in U.S. Pat. No. 6,518,412. The peptides in this patent mimic the same activity as the entire Apo AI protein. They remain in the bloodstream, outside of the cells. Therefore, they do not interact with any cellular material. The Dasseux patent could use the peptide for the treatment of disorders associated with dyslipoproteinemia, and other disorders such as septic shock.

A method for identifying, screening and treating subjects who are at risk of developing or having CVD is disclosed in U.S. Pat. No. 7,378,396 by Hazen et al. The invention in Hazen et al. uses agents that inhibit binding of myeloperoxidase (“MPO”) to a molecule comprising MPO binding site of Apo AI. The agent uses at least four contiguous amino acids of the Apo AI peptide, or a modified form thereof.

While all the inventions mentioned above are directed to various methods to treat CVD, they fail to achieve the advantages offered by our present invention. Only a few studies have been conducted on the role of the propeptide in Apo AI (Jauhiainen et al. J. Lipid Res. 2000; 41: 1872-82; McLeod et al. Biochem. J. 1994; 302: 641-648). All of these studies have involved comparing the serum function of the mature Apo AI protein to the proprotein with the extra six amino acids still attached. These studies have determined that both forms of the proteins (a) can remove cholesterol from cells (however, the presence of the pro form results in a more delipidated HDL particle which may be more efficient at cholesterol removal), (b) can activate LCAT activity, and (c) can transfer cholesterol to an LDL particle. To date, no studies have been performed on just the six amino acid peptide to determine its biological function.

The present invention demonstrates that the six amino acid propeptide may be biologically active, and may have a role in the regulation of cholesterol in the body. This peptide may be a signal to the liver to increase the amount of LDL receptors on the cell surface, resulting in a decrease of cholesterol in the bloodstream, a decrease of cholesterol deposits in the capillaries, and a decrease in the biosynthesis of cholesterol in the liver. It may also signal the adipose cells to increase their uptake of cholesterol.

This propeptide has been conserved throughout evolution by all mammals; however, the sequence does vary from species to species. There is an enzyme located in the serum of all these species to specifically remove this propeptide from the protein. There is a consensus sequence present in all known Apo AI proteins (see Table 1) that looks to be necessary for enzyme specificity and binding. The underlined amino acids represent the propeptide.

TABLE 1

| | Fragment of | |
|---|---|---|
| Organism | Apo AI protein | Propeptide |
| Human being | rhfwqqdepqsq pwdrvkdlat | rhfwqq |
| Norway Rat | wefwqqdeppqs qwdrvkdfat | wefwqq |
| Macaque | rhfwqqdeppqt pwdrvkdlvt | rhfwqq |
| Pig | rhfwqqddpqsp wdrvkdfat | rhfwqq |
| Mouse | whvwqqdepqsq wdkvkdfan | whvwqq |

The fact that both the propeptide and the enzyme that cleaves the protein have been conserved during evolution, suggests that the proprotein and the mature protein differ in their respective functions. A low level of cholesterol inside a liver cell will cause an increase in LDL receptors and will also activate the key regulatory enzyme in cholesterol biosynthesis HMG-CoA reductase. When the liver cell already has a sufficient level of cholesterol, the Apo AI propeptide may serve as a feedback signal to the liver indicating that additional cholesterol from peripheral cells needs to be deposited back into the liver for redistribution or removal. The peptide could accomplish this through transcriptional regulation, by affecting enzyme activity, by affecting the level of LDL receptors on the cell surface, or by affecting the ability of HDL to deposit the cholesterol directly into a liver or adipose cell. Additionally, the peptide may be a signal for the cell to upregulate the amount of apo-lipoprotein AI present in the serum.

Another prominent disease that afflicts approximately twenty million people worldwide is dementia that results in Alzheimer's disease. This is a progressive neurodegenerative disorder that is characterized by the formation of senile plaques and neurofibrillary tangles containing amyloid beta peptide. These plaques damage the neuronal architecture in the limbic areas, including the hippocampus, thus interfering and subsequently crippling the memory process.

In U.S. Application No. 6,682,888, Loring et al. disclosed pharmaceutical compositions comprising several cDNAs for use in detecting changes in expression of genes encoding proteins that are associated with Alzheimer's disease. The cDNAs are expressed differentially in the brain cells or tissues of patients suffering from Alzheimer's disease.

In the study of Alzheimer's disease, there has been great interest in targeting the liver X receptor (“LXR”). LXRα and LXRβ are members of the nuclear receptor superfamily of transcription factors. LXRβ is expressed ubiquitously in cells throughout the human body with particularly high levels in the developing brain, whereas, LXRα predominates in metabolically active tissues such as the liver, small intestine, kidney, macrophages and adipose tissue. Both of these receptors function as intracellular sensors in the event of the presence of an excess amount of intracellular cholesterol and are responsible for regulating genes involved in cholesterol homeostasis (Fan et al. Proc Natl Acad Sci USA. 2008; 105: 13445-13450). The natural ligands for these receptors are thought to be oxidized derivatives of cholesterol (oxysterols) (Janowski et al. Nature. 1996; 383: 728-731). Activation of these receptors has been reported to decrease intestinal cholesterol absorption efficiency (Berge et al. Science. 2000; 290: 1771-1775; Repa et al. Science. 2000; 289: 1524-1529), induce cholesterol efflux from macrophages (Repa et al. Science. 2000; 289: 1524-1529; Costet et al. J. Biol. Chem. 2000; 275: 28240-28245; Schwartz et al. Biochem. Biophys. Res. Commun. 2000; 274: 794-802), and inhibit inflammatory gene expression in a signal-specific manner (Joseph et al. Nat. Med. 2003; 9: 213-219) by decreasing the induction of classical inflammatory genes such as iNOS, COX-2, MMP-9 and various chemokines in response to LPS, TNF-α and IL-1β stimuli. However, activation of the LXRα receptor seems to result in an undesirable elevation of triglyceride levels (Schultz et al. Genes Dev. 2000; 14: 2831-2838), a result that does not seem to occur when only LXRβ is activated (Lund et al. Biochem. Pharmacol. 2006; 71:453-463; Quinet et al. Mol. Pharmacol. 2006; 70: 1340-1349). Therefore, LXRβ specific ligands are currently being pursued as a treatment of atherosclerosis.

Apolipoprotein E (“Apo E”) is a component of “HDL” and is involved in lipoprotein clearance, cholesterol redistribution, and is closely linked to the pathogenesis of Alzheimer's disease. It has been reported that LXR ligand activation leads to an increase in Apo E protein expression and secretion in the hippocampus and vertebral cortex of mice (Liang et al. J Neurochem. 2004; 88: 623-634). The Apo E gene on chromosome 19 has three common alleles (E2, E3, E4), which encode three major Apo E isoforms. It is known that the frequency of Apo E4 allele is markedly increased in sporadic (Poirier et al. Lancet 1993; 342: 697-699) and late onset familial Alzheimer's Disease (Corder et al. Science 1993; 261: 921-923).

One method for the treatment of Alzheimer's disease, based on the Apo E protein, is disclosed by Poirier in U.S. Pat. No. 5,935,781. Here, the method comprises determining the presence of Apo E2 and Apo E3 allele or the absence of Apo E4 allele from peripheral tissues of the patient. This is indicative of the degree of impairment in brain acetylcholine synthesis and nicotinic receptor activity. An afflicted patient is administered a suitable therapeutic agent containing a cholinomimetic drug that improves cognitive performance. The amount of the drug depends on the degree of impairment.

Accumulation of β-amyloid peptide (“Aβ”) in the brain regions responsible for memory and cognitive functions is a neuropathological hallmark of Alzheimer's disease. Cholesterol may be involved in many aspects of Aβ metabolism. It affects generation, aggregation, and clearance of Aβ in the brain. Not only the amount but also the distribution of cholesterol within cells appears to modulate Aβ biogenesis. ACAT is an endoplasmic reticulum (“ER”)-resident enzyme that regulates subcellular cholesterol distribution by converting membrane cholesterol to cholesteryl esters for storage and transport. Studies have shown that inhibition of ACAT strongly reduces Aβ generation and protects from amyloid pathology. (Huttunen et al. Neuoro-degenerative Diseases 2008; 5: 3-4). Thus, an agent that is capable of activating ACAT on the cell surfaces would be useful in treating Alzheimer's disease.

The present invention provides a solution to these problems and issues raised in the prior art. It provides a short peptide that is helpful in regulating HDL, can bind to LXR and can activate ACAT. The invention itself is based on the fundamental discovery that the Apo AI propeptide can enter the nucleus of mammalian cells and bind to proteins within the cell. This is evidence that the propeptide is a biologically active peptide and that this peptide is capable of binding to the LXRβ nuclear receptor protein. The peptide in this invention activates ACAT on the cell surfaces. The peptide is only the six amino acids of the Apo AI propeptide. Based upon the experiments, this peptide enters the nucleus of cells and interacts with the LXRβ nuclear receptor protein.

A key advantage of the invention is that this short peptide is able to enter the nucleus and bind to proteins such as LXRβ, thereby allowing them to play a role as a transcription factor. This is in sharp contrast to the related peptides that are described in the prior art, which are incapable of entering the nucleus.

Another key advantage of the invention is that the peptide of this invention activates ACAT on the cell surface.

Another advantage of the invention is that the peptide is a natural component of human serum, so there should not be any harmful side effects associated with the use of the peptide as a drug. There seems to be enough evidence to date that this peptide does indeed serve a biological function.

A further advantage of the invention is that the short length of the peptides of the invention makes them easily synthesizable by methods well known in the art.

Another advantage of the short peptides disclosed herein is that they may be administered orally instead of by injection, as would be the case with longer peptide sequences in the prior art.

The results disclosed herein indicate that there is an enormous potential for the use of the Apo AI propeptide as a drug in the treatment of disorders associated with dyslipoproteinemia, including hypercholesterolemia, cardiovascular disease, atherosclerosis, restenosis, and other disorders such as septic shock. This peptide may further be used as a drug in the treatment of hyperlipidemia, inflammation (arthritis), and Alzheimer's disease. This invention is therefore a vast improvement over the prior art and presents a novel and effective method for the treatment of some critical diseases that are currently afflicting millions of people of all ages, all over the world.

### SUMMARY OF THE INVENTION

The present invention relates generally to a peptide which is a fragment of the mammalian apo-lipoprotein AI peptide. More precisely, the peptide of the invention comprises the sequence RHFWQQ (human, macaque and pig), the sequence WEFWQQ (rat), and the sequence WHVWQQ (mouse), or suitable modifications thereof, or equivalent synthetic formulations. The present invention is based on the discovery that the aforementioned Apo AI propeptides can enter the nucleus of cells and bind to proteins within the cell. This is evidence that the propeptides are biologically active peptides and that they are capable of binding to the LXR beta nuclear receptor protein.

Additionally, the peptide of this invention also activates ACAT on the cell surface.

The short length of the peptides of the invention makes them easily synthesizable by methods well known in the art. A major advantage of the invention is that this short peptide is able to enter the nucleus and bind to proteins such as LXRβ, thereby allowing them to play a role as a transcription factor.

A pharmaceutical composition or medicament may be therefore prepared comprising the peptides of this invention, or a fragment thereof, along with at least one pharmaceutically acceptable carrier or excipient. Such a pharmaceutical composition may then be used in the treatment of dyslipoproteinemia (hypercholesterol, cardiovascular disease, atherosclerosis, restenosis, and septic shock), inflammation (arthritis), and Alzheimer's disease.

The pharmaceutical compositions of the invention may be administered orally or parenterally by the subcutaneous, intramuscular, or intravenous route. However, a potential advantage of the short peptides disclosed herein is that they may be administered orally instead of by injection, as would be required in the case of longer peptide sequences.

The compositions may be formulated according to techniques and procedures well known in the art and widely described in the literature, and may comprise any of the known carriers, dilutents, or excipients. The compositions may also contain adjuvants, such as buffers, preservatives, dispersing agents, agents that promote rapid onset of action, or prolonged duration of action, and the like.

These and other features, variations, and advantages which characterize this invention, will be apparent to those skilled in the art, from a reading of the following detailed description and a review of the associated drawings.

All features and advantages of this invention will be understood from the detailed descriptions provided. This description, however, is not meant to limit the embodiments, and merely serves the purpose of describing certain embodiments in detail.

### BRIEF DESCRIPTION OF THE DRAWINGS

A better understanding of the present invention will be had upon reference to the following description in conjunction with the accompanying drawings, wherein:

FIG. 1 is a confocal image of Zucker rat liver cells incubated with the apolipoprotein AI propeptide WEFWQQ. The peptide is labeled at the N-terminus with fluorescein. The propeptide is clearly entering the cells and entering the nucleus of the cells;

FIGS. 2A and 2B show confocal images of human epithelial cells incubated with the apolipoprotein AI propeptide WEFWQQ. The peptide is labeled at the N-terminus with fluorescein. The cells were visualized at the fluorescent wavelength for fluorescein (FIG. 2A) and the fluorescent wavelength of LDS-751 (FIG. 2B). LDS-751 is a known nuclear staining molecule;

FIG. 3 is a confocal image of a human epithelial cell incubated with the methylated apolipoprotein AI propeptide WEFWQQ. The peptide is labeled at the N-terminus with fluorescein. The propeptide is clearly entering the cell and the nucleus of the cell. The propeptide is localized to specific areas within the cytosol;

FIGS. 4A and 4B show Rat liver separated by native polyacrylamide gel electrophoresis (“PAGE”): FIG. 4A before incubation with fluorescent propeptide; FIG. 4B after incubation with propeptide. Lanes 1, 2, 3 are molecular weight standards, concentrated lysate, and dilute lysate, respectively. Arrows indicate new band appearing after incubation;

FIG. 5 shows Rat and mouse tissue separated by native PAGE after incubation with fluorescent propeptide. Lanes 1 and 3 are old Rat tissue lysate. Lanes 2 and 4 are young Mouse tissue lysate. The top arrow indicates the same band seen as FIG. 4B (see the arrows). The bottom arrow indicates an additional protein bound to the propeptide;

FIG. 6 shows Rat adipose and liver proteins isolated from the Apo AI peptide beads and separated by sodium dodecyl sulfate (“SDS”) PAGE. The protein band highlighted was excised from the gel, digested with trypsin, and identified through peptide mass fingerprint mapping.

### DETAILED DESCRIPTION OF THE INVENTION

While the invention will be described in connection with certain embodiments, there is no intent to limit it to these embodiments. On the contrary, the intent is to cover all alternatives, modification and equivalents as included within the spirit and scope of the invention. Various changes may be made to the function and arrangement of the elements described herein, without changing the scope of the invention being disclosed. It should be noted that the following description serves to teach at least one instance of how the various elements may be arranged to achieve the stated goals of this invention.

Unless otherwise defined, all technical and scientific terms used herein have the same meaning as commonly understood by one of ordinary skill in the art to which this invention belongs. The terminology used in the description is used for describing particular embodiments and is not intended to be limiting of this invention. As used in the description of this invention and the appended claims, the singular forms “a”, “an”, and “the” are intended to include the plural forms as well, unless the context clearly indicates otherwise.

The term “peptide” includes analogues and fragments thereof. The term “analogue” refers to any derivative of the peptide or peptides in which one or more amino acids have been substituted with amino acids of similar size and charge. Analogues also include peptides which contain one or more amino acids in an altered configuration.

“Derivative” refers to a protein that has been subjected to a chemical modification such as replacement of a hydrogen group by, for example, an acetyl, acyl, alkyl, amino, formyl, or morphalino group. Derivative peptides and nucleotides may encode proteins that retain the essential biological characteristics of naturally occurring proteins.

“Fragment” refers to a chain of consecutive nucleotides from the Apo AI peptide, or the Apo AI propeptide, that comprises fewer amino acids than the full-length Apo AI. Fragments may be used to identify related nucleic acid molecules and to screen for and purify a ligand. Nucleic acids and their ligands that are identified thus are useful in regulating replication, transcription, or translation.

The terms “functional equivalent” to an Apo AI peptide, or to the Apo AI propeptide as used herein, refers to a gene product that exhibits one of the biological activities of an Apo AI peptide or propeptide, including entering the cell, binding to lipids, formation of HDL-like complexes, activation of ACAT, increasing serum HDL concentration, and promotion of cholesterol efflux.

“Ligand” refers to any agent, molecule, or compound which will bind complementarily to a complementary site on a DNA strand, polynucleotide, or protein, and may be composed of at least one of the following: inorganic and organic substances including nucleic acids, proteins, carbohydrates, fats, and lipids.

“Purified” or “isolated” refers to any molecule or compound that is separated from its natural environment and is at least about 60% free, preferably about 75% free, and most preferably, about 90% free, from other compounds with which it is naturally associated. Purified does not require absolute purity; rather, it is intended as a relative term. A purified peptide preparation may thus be understood to be one in which the peptide or protein is more enriched than the peptide or protein is in its natural environment within a cell.

The term “treating” when used with respect to “treating” a subject with CVD or Alzheimer's disease, or any of the other disorders discussed herein, refers to a reduction, prevention, or elimination of one or more symptoms, complications, or manifestations characteristic of the particular disorder being treated. This is generally well understood to a person of ordinary skill in the art.

The term “therapeutically effective amount,” when used with respect to the quantity of the relevant peptide in a pharmaceutical preparation, will mean any amount sufficient to show a meaningful clinical benefit without toxicity. This amount may vary with the particular form of the peptide used, and the route of administration. It is well understood to those skilled in the art that such amount may be suitably adjusted to achieve the desired results. Toxicity and therapeutic efficacy are usually determined using standard pharmaceutical procedures in cell culture and experimental animals. All these factors are to be ultimately determined in clinical studies.

The core peptide of the invention comprises the sequence RHFWQQ (human beings (UniProt accession number PO₂₆₄₇: www.uniprot.org/uniprot/P02647) (SEQ ID NO: 3), macaque (UniProt accession number P68292: www.uniprot.org/uniprot/P68292) (SEQ ID NO: 24), and pig (UniProt accession number P18648: www.uniprot.org/uniprot/P18648)) (SEQ ID NO: 31), or a fragment thereof, the sequence WEFWQQ (rat (UniProt accession number P04639: www.uniprot.org/uniprot/P04639)) (SEQ ID NO: 17), or a fragment thereof, and the sequence WHVWQQ (mouse (UniProt accession number Q00623: www.uniprot.org/uniprot/Q00623)) (SEQ ID NO: 38), or a fragment thereof. Suitable modifications or equivalent synthetic formulations of these peptides are also within the scope of the claimed invention. These sequences are the amino acid sequences of the corresponding Apo AI propeptides.

In one embodiment of the present invention, the nucleotide sequences encode core peptides, or their analogues, that have the following structural formula: X₁-X₂-X₃-X₄-X₅-X₆-X₇-X₈-X₉-X₁₀ (“Structural Formula I”) wherein:

X₁ is Arg (R);

X₂ is His (H);

X₃ is Phe (F);

X₄ is Trp (W);

X₅ is Gln (Q);

X₆ is Gln (Q);

X₇ is Asp (D);

X₈ is Glu (E);

X₉ is Pro (P), His (H), or Arg (R);

X₁₀ is Pro (P) or Arg (R).

The symbol “-” usually represents a peptide bond or amide linkage. Peptides that are functionally equivalent to structural formula I are within the scope of this invention.

Some illustrative nucleotide sequences of this aspect of the invention are those that encode core peptides selected from the group consisting of:

| | | |
|---|---|---|
| peptide 62 | RHFWQQDEPP; | (SEQ ID NO: 62) |
| peptide 63 | RHFWQQDEHP; | (SEQ ID NO: 63) |
| peptide 64 | RHFWQQDERP; | (SEQ ID NO: 64) |
| peptide 65 | RHFWQQDEPR; | (SEQ ID NO: 65) |
| peptide 66 | RHFWQQDEHR; | (SEQ ID NO: 66) |
| peptide 67 | RHFWQQDERR. | (SEQ ID NO: 67) |

In another embodiment of the present invention, the nucleotide sequences encode core peptides, or their analogues, that have the following structural formula: X₁-X₂-X₃-X₄-X₅-X₆-X₇-X₈-X₉-X₁₀ (“Structural Formula II”) wherein:

X₁ is Trp (W);

X₂ is Glu (E);

X₃ is Phe (F);

X₄ is Tip (W);

X₅ is Gln (Q);

X₆ is Gln (Q);

X₇ is Asp (D);

X₈ is Glu (E);

X₉ is Pro (P);

X₁₀ is Gln (Q).

The symbol “-” usually represents a peptide bond or amide linkage. Peptides that are functionally equivalent to structural formula II are within the scope of this invention.

An illustrative nucleotide sequence of this aspect of the invention is that which encodes the core peptide:

| | | |
|---|---|---|
| peptide 68 | WEFWQQDEPQ | (SEQ ID NO: 68) |

In a further embodiment of the present invention, the nucleotide sequences encode core peptides, or their analogues, that have the following structural formula: X₁-X₂-X₃-X₄-X₅-X₆-X₇-X₈-X₉-X₁₀ (“Structural Formula III”) wherein:

X₁ is Trp (W);

X₂ is His (H);

X₃ is Val (V);

X₄ is Trp (W);

X₅ is Gln (Q);

X₆ is Gln (Q);

X₇ is Asp (D);

X₈ is Glu (E);

X₉ is Pro (P);

X₁₀ is Gln (Q).

The symbol “-” usually represents a peptide bond or amide linkage. Peptides that are functionally equivalent to structural formula III are within the scope of this invention.

An illustrative nucleotide sequence of this aspect of the invention is that which encodes the core peptide:

| | | |
|---|---|---|
| peptide 69 | WHVWQQDEPQ | (SEQ ID NO: 69) |

While structural formula I, II and III contain 10 specified amino acids, it is to be understood that the core peptides of this invention can be 4-10 amino acids in length. These would include shorter sequences where at least one and up to six of residues X₁, X₂, X₇, X₈, X₉, and X₁₀ are deleted, and that retain the ability to enter the nucleus of a cell, bind to the DNA, bind to a protein bound to a DNA, or activate ACAT on the cell surface. The shorter sequences are also understood to be contiguous. In this context, contiguous means that the residue numbering is continuous. For instance, if X₂ is deleted, then X₁ must also be deleted. Similarly, if X₇ is present in the sequence, then so must X₈, X₉, and X₁₀.

The present invention is based on the discovery that the aforementioned Apo AI propeptides can enter the nucleus of a cell and bind to proteins within the cell. This is evidence that the propeptides are biologically active peptides and that they are capable of binding to the LXR beta nuclear receptor protein.

Additionally, the peptide of this invention also activates ACAT on the cell surface.

The invention embodies and provides for such peptides for use in therapy and more importantly for use in the treatment of disorders associated with dyslipoproteinemia, including hypercholesterol, cardiovascular disease, atherosclerosis, restenosis, and septic shock, along with inflammatory diseases and Alzheimer's disease. The present invention also contemplates and embodies the peptide sequences being combined with therapeutic and other pharmaceutical agents and additives into a pharmaceutical composition comprising a peptide of the invention together with at least one pharmaceutically acceptable carrier or excipient.

The present invention provides the use of the peptides of the invention in the manufacture of a medicant for combating and treating various disease states and processes, including but not limited to atherosclerosis and Alzheimer's Disease. “Non-native” isomers of the “native” L-amino acid peptide sequences are also contemplated by the inventor for use within the scope of the invention. Also included within the scope of the invention are derivatives of the peptides, including but not limited to D-amino acids, acetylated derivatives, methylated derivatives, and oxidized derivatives of the peptides. The terms “D-amino acid” and “L-amino acid” are used to refer to absolute configuration of the amino acid, rather than a particular direction of rotation of plane-polarized light. The invention encompasses peptides having N- and/or C-terminal extensions, or flanking sequences added to the peptide. The length of the extended peptides may vary, but may not be more than ten amino acids long. The present invention also includes the nucleotide sequences of the peptides of the invention.

It is noted that the peptides disclosed in this invention may be chemically modified. In such cases, at least one of the amino acid residues may be modified either by a natural process including post-translational modifications, or by chemical modification techniques which are well known in the art. Examples of chemical modifications include acetylations and glycolysations, glycosamine-glycinations, ADP-ribosylations, covalent attachment of a lipid or lipid derivative, methylation, etc. The peptides of the invention may additionally have a non-peptide component attached, such as polyethylene glycol, carbohydrates or lipid fatty acid conjugates.

The above modifications may be needed for increased stability, bioavailability or activity of the peptides of the invention.

Conversion of linear peptides to cyclic peptide analogs may also be used to improve metabolic stability. It is known in the art that cyclic peptides are much less sensitive to enzymatic degradation (see generally, Veber, et al Nature 1981; 292:55-58).

The short length of the peptides of the invention makes them easily synthesizable by methods well known in the art. The main advantage of the invention is that these short peptides are able to enter the nucleus and bind to proteins such as LXRβ, thereby allowing them to play a role as a transcription factor.

A pharmaceutical composition or medicament may therefore be prepared comprising the peptides of this invention. Such a pharmaceutical composition may then be used in the treatment of dyslipoproteinemia (hypercholesterol, cardiovascular disease, atherosclerosis, restenosis, and septic shock), inflammation (arthritis), and Alzheimer's disease.

The pharmaceutical compositions of the invention may be administered orally or parenterally by the subcutaneous, intramuscular, or intravenous route. However, a potential advantage of the short peptides disclosed herein is that they may be administered orally instead of by injection, which would be required with longer peptide sequences.

The pharmaceutical compositions of the invention may be formulated according to techniques and procedures that are well known in the art and described in the literature. The compositions may comprise adjuvants such as buffers, preservatives, dispersing agents etc.

The peptides of the invention and any protein-based active agents may also be replaced with therapeutic peptides having functionally equivalent activity. Such compounds are well known in the art and may be identified using various molecular libraries, and synthesized using standard techniques. Such standard techniques may be used to obtain the peptidomimetic compounds according to the present invention, namely peptidomimetic organic compounds which show substantially similar or the same biological activity as the peptide of the invention.

The Apo AI propeptide may bind to the LXRβ receptor at the same site as oxysterols. The peptide does contain a Tryptophan residue at position 4 and a nonpolar amino acid residue at position 3 that seem to be conserved in all mammals. Tryptophan contains a hydrophobic ring structure that may fit in the hydrophobic binding pocket of LXRβ. Alternatively, the peptide may bind at an alternate site and enhance oxysterol binding. The binding of the Apo AI propeptide may also have an impact on the binding partner (LXRβ forms dimers with other nuclear receptors like RXR) of the LXRβ receptor, or it may have an effect on the DNA binding site thus altering gene expressions. The binding of the peptide may result in an increased expression of apolipoprotein AI in the liver cell, thus increasing the serum levels of Apo AI and HDL. The binding may also result in an increased level of LDL receptors and scavenger receptor class B type I receptors (a cell surface HDL receptor) on liver cell surfaces.

Studies have indicated that the intestine may play a role as an excretory organ in reverse cholesterol transport (Kruit et al. Gastroenterology 2005; 128:147-156; Plosch et al. J. Biol. Chem. 2002; 277:33870-33877). In this case, it would be important for the intestine to know that the cholesterol is not needed and should be excreted. The Apo AI propeptide may provide this necessary control for cholesterol excretion by indicating the level of cholesterol in the body.

The specific structure and function of the core peptides or analogues of this invention may be assayed in order to select the biologically active peptides. Thus, we may assay the core peptides or their analogues for their ability to enter the nucleus of a mammalian cell, or their ability to bind to the DNA, or their ability to bind to a protein bound to a DNA, or their ability to activate ACAT on the cell surface.

Methods and assays for analyzing aforementioned structural or functional properties of the peptides are well known in the art. Some preferred methods are described in the following examples.

### Labeled Peptide

A peptide with the amino acid sequence WEFWQQ (corresponding to the rat sequence of the Apo AI propeptide) containing a fluorescein group attached to the N-terminus was custom synthesized by Cambridge biochemical reagents. Synthetic version of the peptide was made using standard Merrifield peptide synthesis techniques (http://en.wikipedia.org/wiki/Peptide_synthesis). Other standard techniques that would apply here would be the use of bacteria to synthesize large quantities of the peptide (http://en.wikipedia.org/wiki/Recombinant_DNA).

### Acquisition of Liver Cells for Confocal Experiments

Liver tissue was obtained from lean Zucker rats and placed in liver digest media. The liver digest media was then drained into a conical vial and the liver was placed in another sterile beaker of hepatozyme wash media. Clean gauze was placed at the opening to the conical vials and the liver was washed with the hepatozyme wash media yet again. This process filtered the liver cells into the conical vials. The cells were centrifuged for five minutes at 6000 rpm. The cells were washed thoroughly with hepatozyme wash media and then resuspended in 5% Fetal Bovine Serum, 2 mM glutamine hepatozyme wash medium and pipetted into collagen wells. The collagen well plates were incubated at 37° Celsius and 5% CO₂. After 19 hours, the media was replaced with clean, fresh Hepatozyme-SFM (3 mL). The media was further supplemented with L-glutamine (2 mM) after 23 hours. The collagen bottom was cut out of one of the wells for viewing on the confocal microscope. This piece of the collagen well was divided into several small pieces. The cells were maintained in media and then treated with the fluorescently labeled propeptide and observed on the microscope (40× oil). Confocal studies were also run on simple human squamous epithelial cells; which were obtained through a cheek swab. The liver and the epithelial cells were also treated with methylated propeptide which contained a methyl group at the C-terminus and on the glutamic acid residue.

### Acquisition of Liver Samples for Modified Western Blot Experiments

Rat liver tissue from an old (23 months) rat and mouse liver tissue from a young mouse (3 months) was obtained from Dr. Eric R. Blough (Biology department, Marshall University, Huntington, W. Va.). 0.104 g of liver tissue from the young mouse and 0.20 g of tissue from the old rat were placed in separate microcentrifuge tubes and washed thoroughly with phosphate buffered saline (“PBS”).

### Cell Lysis

Rat liver tissue was lysed using CellLytic-MT lysis buffer (Sigma) following the procedure provided from Sigma. Along with the lysis buffer, a protease inhibitor cocktail containing pepstatin, aprotinin, and leupeptin was added. The samples were then centrifuged for 15 minutes at 14,000 rpm. Supernatant containing the cellular proteins was pulled from the samples and centrifuged for another 10 minutes.

### Native PAGE

Proteins from the lysate were separated by Native PAGE on a 12% polyacrylamide gel. The gel was run using Tris/Glycine running buffer for one hour at 200 V.

### Peptide Incubation

After protein separation by PAGE was complete, a picture of the gel was taken using an ultraviolet (“UV”) lamp to excite any fluorescent molecules. The UV exposure time was 20 s. The gel was then incubated with the fluorescent labeled Apo AI propeptide in Tris buffered saline with 5% Tween (“TBST”) overnight on a rocking platform. Another picture of the gel was taken using the same UV exposure time as the initial picture.

### Peptide Beads

The Apo AI propeptide was attached to benzyloxybenzylbromide resin at its N-terminus. Rat liver tissue was lysed using CellLytic-MT lysis buffer using the procedure outlined above. This buffer may be purchased from Sigma-Aldrich Corp. (“Sigma”) (St. Louis, Mo.). Benzyloxybenzyl bromine resin was added to the protein lysate, and the sample was shaken for one hour in order to preclear the lysate of proteins that had a binding affinity to the resin itself. The protein lysate was removed and added to the peptide modified beads. The solution was shaken for one hour; the supernatant was then removed leaving only the resin beads. The resin beads were washed three times with PBS, and then 30 μl of gel loading solution was added to the resin beads. The solution was heated at 100° C. for five minutes and then loaded onto a polyacrylamide gel (4-20% polyacrylamide) and separated by SDS-PAGE.

### In-Gel Digestion

Protein bands of interest were cut from the gel and digested with trypsin overnight following the procedure used at University of California, San Francisco (http://donatello.ucsf.edu/ingel.html). The peptide fragments were analyzed on a Bruker MALDI-TOF mass spectrometer which provided the peptide fragment molecular weight maps of the proteins for protein identification.

### EXAMPLES

### Example 1

### The Propeptide is Capable of Entering the Nucleus

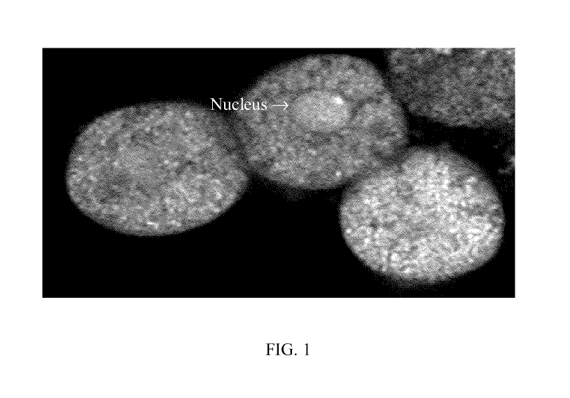

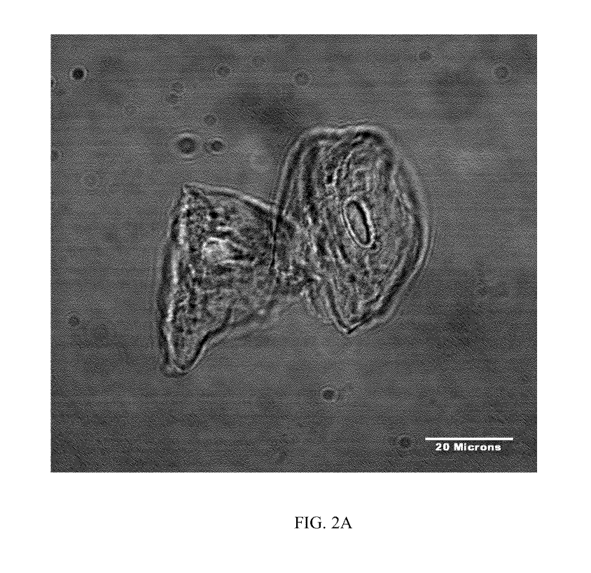

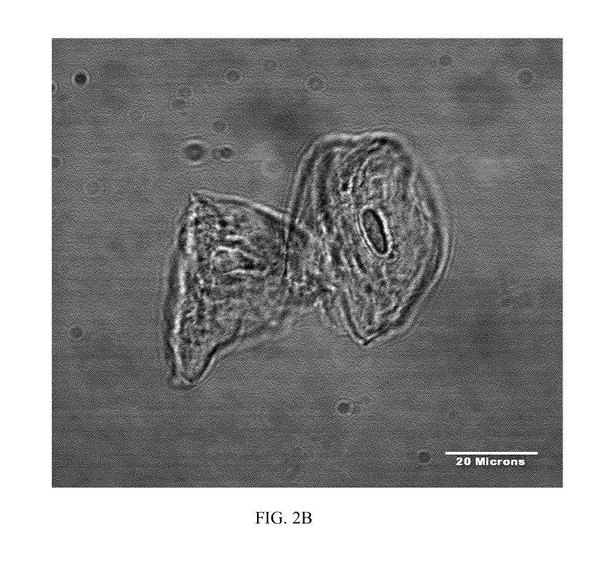

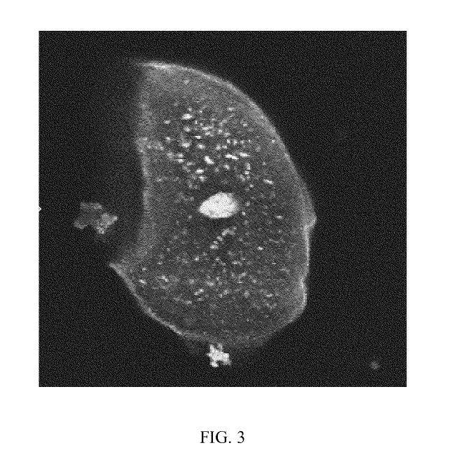

Liver and epithelial cells were treated with the fluorescently labeled apolipoprotein AI propeptide, WEFWQQ, and observed by confocal microscopy (FIGS. 1, 2A and 2B). The cell shown in FIG. 2B was also treated with LDS-751, a known nuclear staining molecule. In addition, the cells were also treated with the methylated version of the peptide (as seen in FIG. 3). In all cases, the peptide could be seen entering the cells within seconds of peptide application. FIG. 3 shows the confocal image of a human epithelial cell treated with the methylated peptide. The peptide is distributed in distinct compartments within the cytosol indicating that the peptide may be interacting with a protein located within these compartments. The peptide enters the nucleus of the cell within one minute of application. Substances do not typically enter the nucleus of cells unless they are specifically targeted to do so. This is the cell's way of protecting the DNA located within the nucleus. Therefore, these results imply that the peptide does indeed have a biological function within the cell that may include transcriptional activity.

### Example 2

### The Propeptide is Capable of Binding Directly to the DNA or to a Protein Bound to the DNA

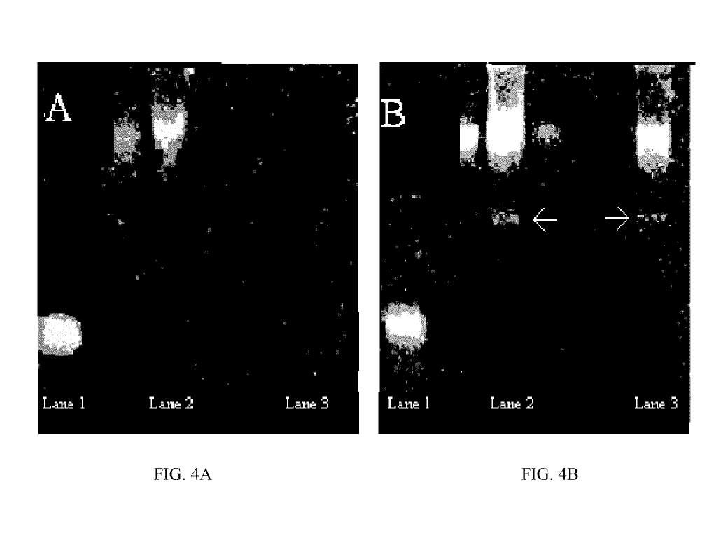

A set of experiments were conducted to determine whether the peptide was interacting with any proteins within the cell. We decided to do a modified Western Blot experiment using the fluorescent peptide in place of an antibody. Liver tissue from an old rat (23 months) was lysed and the total cellular proteins were separated on a Native PAGE gel. The gel was then incubated overnight with the fluorescently labeled Apo AI propeptide WEFWQQ. FIG. 4 shows the gel before (4A) and after (4B) incubation with the peptide. Both images were obtained using the same UV exposure time. The arrow indicates where a new fluorescent band emerges after incubation with the peptide. This would indicate that the peptide is binding to a protein on the gel. We do not believe that this protein is simply albumin because an albumin standard in lane 1 did not migrate to the same location in the gel as the unknown protein. We have excised this protein band from the gel and digested the protein with trypsin in preparation for mass spectral protein identification. We also saw an increase in the fluorescence intensity of a higher molecular weight band after peptide incubation. The fact that the higher band fluoresces even without added peptide (FIG. 4A) indicates that this may be DNA. Therefore, the peptide may bind directly to DNA or to a protein that is bound to DNA. This could be a significant result in that the peptide may undertake a role as a transcription factor.

### Example 3

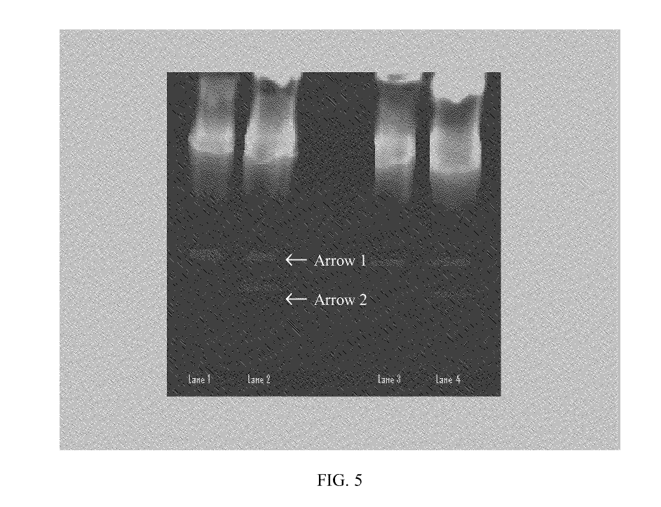

The experiment in Example 2 was repeated using liver tissue from the old rat as well as liver tissue from the young mouse. The Native gel after incubation with the peptide is shown in FIG. 5. Lysate from the old rat liver was loaded into lanes 1 and 3, and lysate from the young mouse liver was loaded into lanes 2 and 4. In FIG. 5 Arrow 1, we again see the same band as was seen in FIG. 4 in both the rat and mouse samples.

### Example 4

### The Propeptide is Capable of Binding to LXRβ

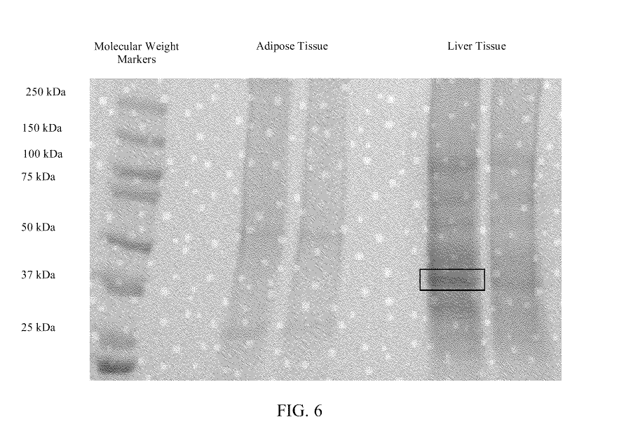

Another set of experiments were designed to determine the identity of any proteins capable of binding to the Apo AI propeptide. The Apo AI propeptide, WEFWQQ, was attached to benzyloxybenzylbromide resin beads at its N-terminus. Liver tissue from a lean Zucker rat was lysed, and the supernatant was incubated with benzyloxybenzylbromide resin beads (no peptide attached) in order to preclear the liver lysate of all protein capable of binding to the resin beads themselves. The lysate was then incubated with the peptide beads for one hour. After one hour, the supernatant was removed, and the resin beads were washed three times in PBS in order to remove all nonspecific binding proteins. Then, 30 μl of gel loading buffer was added to the beads, and the solution was heated for five minutes at 100° C. The sample was then loaded onto a 4-20% acrylamide gel, and the proteins were separated by SDS-PAGE. Proteins were stained with Coommassie Blue staining solution. The gel is shown in FIG. 6. There were several faint protein bands that can be seen in this gel, but the darkest, most concentrated band can be seen in the liver tissue lane at about 45 kDa. This protein band was excised from the gel and digested overnight with trypsin. The molecular weights of the resultant tryptic peptide fragments were obtained on a MALDI-TOF time of flight mass spectrometer. A Mascot database search of these molecular weights identified the protein as the oxysterol receptor LXRβ.

### Example 5

### A Pharmaceutical Composition

The peptide of this invention may be added directly to saline solution for injection or intravenous administration. To prepare 10 mL of the composition, mix 15 mg of the propeptide with 25 mg of M-Kresol and 160 mg of glycerol and add water and either 10% hydrochloride acid or 10% sodium hydroxide to make 10 mL of a solution with a pH of 7.0-7.8.

There are additional protein bands present in the gel (FIG. 5) that have not been identified. These bands may be proteins that are nonspecifically attracted to the resin beads. Although a preclear step of the tissue lysate with the beads was performed, there still may have been some left in the lysate. These bands could also be additional proteins that interact with the Apo AI propeptide. Therefore, the biological activity of this propeptide may not be limited to its interaction with LXRβ.

While many novel features have been described above, the invention is not limited to these physical embodiments. It is described and illustrated with particularity so that that those skilled in the art may understand all other embodiments that may arise due to modifications, changes in the placement of the relative components, omissions and substitutions of the embodiments described herein, that are still nonetheless within the scope of this invention.

### REFERENCES

1. Lewis, et al. Circ Res. 2005; 96: 1221-32.
2. Rubin, et al. Nature. 1991; 353: 265-7.
3. Plump, et al. Proc Natl Acad Sci USA. 1994; 91: 9607-11.
4. Jauhiainen, et al. J. Lipid Res. 2000; 41: 1872-82.
5. McLeod, et al. Biochem. J. 1994; 302: 641-648.
6. Janowski, et al. Nature. 1996; 383: 728-731.
7. Berge, et al. Science. 2000; 290: 1771-1775.
8. Repa, et al. Science. 2000; 289: 1524-1529.
9. Costet, et al. J. Biol. Chem. 2000; 275: 28240-28245.
10. Schwartz, et al. Biochem. Biophys. Res. Commun. 2000; 274: 794-802.
11. Joseph, et al. Nat. Med. 2003; 9: 213-219.
12. Schultz, et al. Genes Dev. 2000; 14: 2831-2838.
13. Lund, et al. Biochem. Pharmacol. 2006; 71: 453-463.
14. Quinet, et al. Mol. Pharmacol. 2006; 70: 1340-1349.
15. Liang, et al. J Neurochem. 2004; 88: 623-634.
16. Poirier, et al. Lancet 1993; 342: 697-699.
17. Corder, et al. Science 1993; 261: 921-923.
18. Huttunen, et al. Neuoro-degenerative Diseases 2008; 5: 3-4.
19. Veber, et al Nature 1981; 292: 55-58.
20. Kruit, et al. Gastroenterology 2005; 128: 147-156.
21. Plosch, et al. J. Biol. Chem. 2002; 277: 33870-33877.
22. Fan, et al. Proc Natl Acad Sci USA. 2008; 105: 13445-13450.
23. UniProt accession number P02647: www.uniprot.org/uniprot/P02647.
24. UniProt accession number P68292: www.uniprot.org/uniprot/P68292.
25. UniProt accession number P18648: www.uniprot.org/uniprot/P18648.
26. UniProt accession number P04639: www.uniprot.org/uniprot/P04639.
27. UniProt accession number Q00623: www.uniprot.org/uniprot/Q00623.
28. http://en.wikipedia.org/wiki/Peptide_synthesis.
29. http://en.wikipedia.org/wiki/Recombinant_DNA.
30. http://donatello.ucsf.edu/ingel.html.

I claim:

1. An isolated or purified peptide fragment of Apo-lipoprotein AI from an organism wherein:
said peptide comprises the sequence WQQ;
said peptide is 4-10 amino acids in length;
said peptide has the ability to enter the nucleus of a mammalian cell.

2. The peptide of claim 1, wherein:
said peptide comprises the sequence FWQQ.

3. The peptide of claim 1, wherein:
said peptide undertakes a transcriptor role by binding directly with the DNA.

4. The peptide of claim 1, wherein:
said peptide undertakes a transcriptor role by binding directly with a protein that is bound to the DNA.

5. The peptide of claim 4, wherein:
said protein is LXRβ.

6. The peptide of claim 1, wherein:
said peptide has the ability to activate ACAT on the surface of said cell.

7. The peptide of claim 1, wherein:
said mammalian cell is a human cell.

8. The peptide of claim 2, wherein:
said peptide comprises the sequence RHFWQQ;
said organism is selected from the group consisting of:
(i) Homo Sapiens (SEQ ID NO: 3);
(ii) Macaca (SEQ ID NO: 24); and
(iii) Sus Scrofa. (SEQ ID NO: 31).

9. The peptide of claim 2, wherein:
said organism is of the genus Rattus.

10. The peptide of claim 9, wherein:
said organism is Rattus Norvegicus; and
said peptide comprises the sequence WEFWQQ (SEQ ID NO: 17).

11. The peptide of claim 1, wherein:
said organism is Mus Musculus; and
said peptide comprises the sequence WHVWQQ (SEQ ID NO: 38).

12. The peptide of claim 1, wherein:
at least one of said amino acids is substituted by a non-native isomer of said amino acid.

13. The peptide of claim 1, wherein:
said peptide fragment is a derivative.

14. A nucleotide sequence encoding a peptide fragment of Apo-lipoprotein AI comprising:
(i) 4 to 10-residue peptide or peptide analogue with the structural formula X₁-X₂-X₃-X₄-X₅-X₆-X₇-X₈-X₉-X₁₀
or a pharmaceutically acceptable salt thereof, wherein said structure is selected from the group consisting of:
a. X₁ is Arg (R), X₂ is His (H), X₃ is Phe (F), X₄ is Trp (W), X₅ is Gln (Q), X₆ is Gln (Q), X₇ is Asp (D), X₈ is Glu (E), X₉ is Pro (P), His (H), or Arg (R), and X₁₀ is Pro (P) or Arg (R);
b. X₁ is Trp (W), X₂ is Glu (E), X₃ is Phe (F), X₄ is Trp (W), X₅ is Gln (Q), X₆ is Gln (Q), X₇ is Asp (D), X₈ is Glu (E), X₉ is Pro (P), and X₁₀ is Gln (Q); and
c. X₁ is Trp (W), X₂ is His (H), X₃ is Val (V), X₄ is Trp (W), X₅ is Gln (Q), X₆ is Gln (Q), X₇ is Asp (D), X₈ is Glu (E), X₉ is Pro (P), and X₁₀ is Gln (Q);
(ii) a deleted form of said structural formula in which at least one and up to six of residues X₁, X₂, X₇, X₈, X₉, and X₁₀ are deleted.

15. A nucleotide sequence of claim 14 encoding a peptide fragment of Apo-lipoprotein AI having an amino acid sequence selected from the group consisting of:

| | | |
|---|---|---|
| peptide 62 | RHFWQQDEPP; | (SEQ ID NO: 62) |
| peptide 63 | RHFWQQDEHP; | (SEQ ID NO: 63) |
| peptide 64 | RHFWQQDERP; | (SEQ ID NO: 64) |
| peptide 65 | RHFWQQDEPR; | (SEQ ID NO: 65) |
| peptide 66 | RHFWQQDEHR; | (SEQ ID NO: 66) |
| peptide 67 | RHFWQQDERR; | (SEQ ID NO: 67) |
| peptide 68 | WEFWQQDEPQ; | (SEQ ID NO: 68) |
| and | | |
| peptide 69 | WHVWQQDEPQ. | (SEQ ID NO: 69) |

16. A pharmaceutical preparation comprising a therapeutically effective amount of a peptide fragment of Apo-lipoprotein AI from an organism, wherein:
said peptide comprises the sequence WQQ;
said peptide is 4-10 amino acids in length;
said peptide has the ability to enter the nucleus of a mammalian cell.

17. The peptide of claim 16, wherein:
said peptide comprises the sequence FWQQ.

18. The peptide of claim 16, wherein:
said peptide undertakes a transcriptor role by binding directly with the DNA.

19. The peptide of claim 16, wherein:
said peptide undertakes a transcriptor role by binding directly with a protein that is bound to the DNA.

20. The peptide of claim 19, wherein:
said protein is LXRβ.

21. The peptide of claim 16, wherein:
said peptide has the ability to activate ACAT on the surface of said cell.

22. The peptide of claim 16, wherein:
said mammalian cell is a human cell.

23. The peptide of claim 17, wherein:
said peptide comprises the sequence RHFWQQ;
said organism is selected from the group consisting of:
(i) Homo Sapiens (SEQ ID NO: 3);
(ii) Macaca (SEQ ID NO: 24); and
(iii) Sus Scrofa. (SEQ ID NO: 31).

24. The peptide of claim 17, wherein:
said organism is of the genus Rattus.

25. The peptide of claim 24, wherein:
said organism is Rattus Norvegicus; and
said peptide comprises the sequence WEFWQQ (SEQ ID NO: 17).

26. The peptide of claim 16, wherein:
said organism is Mus Musculus; and
said peptide comprises the sequence WHVWQQ (SEQ ID NO: 38).

27. The peptide of claim 16, wherein:
at least one of said amino acids is substituted by a non-native isomer of said amino acid.

28. The peptide of claim 16, wherein:
said peptide fragment is a derivative.

29. A pharmaceutical preparation comprising a therapeutically effective amount of a peptide fragment of Apo-lipoprotein AI, encoded by a nucleotide sequence, wherein said peptide comprises:
(i) 4 to 10-residue peptide or peptide analogue with the structural formula X₁-X₂-X₃-X₄-X₅-X₆-X₇-X₈-X₉-X₁₀
or a pharmaceutically acceptable salt thereof, wherein said structure is selected from the group consisting of:
a. X₁ is Arg (R), X₂ is His (H), X₃ is Phe (F), X₄ is Trp (W), X₅ is Gln (Q), X₆ is Gln (Q), X₇ is Asp (D), X₈ is Glu (E), X₉ is Pro (P), His (H), or Arg (R), and X₁₀ is Pro (P) or Arg (R);
b. X₁ is Trp (W), X₂ is Glu (E), X₃ is Phe (F), X₄ is Trp (W), X₅ is Gln (Q), X₆ is Gln (Q), X₇ is Asp (D), X₈ is Glu (E), X₉ is Pro (P), and X₁₀ is Gln (Q); and
c. X₁ is Trp (W), X₂ is His (H), X₃ is Val (V), X₄ is Trp (W), X₅ is Gln (Q), X₆ is Gln (Q), X₇ is Asp (D), X₈ is Glu (E), X₉ is Pro (P), and X₁₀ is Gln (Q);
(ii) a deleted form of said structural formula in which at least one and up to six of residues X₁, X₂, X₇, X₈, X₉, and X₁₀ are deleted.

30. The pharmaceutical preparation of claim 16, or 29, in combination with one or more compounds such as:
(i) protein-based active agents;
(ii) pharmaceutically innocuous fillers;
(iii) pharmaceutically innocuous adjuvants.

31. The pharmaceutical preparation of claim 30, wherein:
said compound is a saline solution.

32. The pharmaceutical preparation of claim 30, wherein:
said peptide is substituted with therapeutic peptides with peptidomimetics having functionally equivalent activity.

33. The pharmaceutical preparation of claim 30, wherein:
said protein-based active agent is substituted with therapeutic peptides with peptidomimetics having functionally equivalent activity.

34. The pharmaceutical preparation of claim 30, wherein:
said preparation is used to treat disorders associated with dyslipoproteinemia.

35. The pharmaceutical preparation of claim 34, wherein said disorder is selected from a group consisting of:
(i) hypercholesterol;
(ii) cardiovascular disease;
(iii) atherosclerosis;
(iv) restenosis; and
(v) septic shock.

36. The pharmaceutical preparation of claim 30, wherein:
said preparation is used to treat inflammatory diseases.

37. The pharmaceutical preparation of claim 30, wherein:
said preparation is used to treat Alzheimer's disease.

38. The pharmaceutical preparation of claim 30, wherein:
said preparation is capable of being administered orally.

39. The pharmaceutical preparation of claim 30, wherein:
said preparation is capable of being administered parenterally.

40. The pharmaceutical preparation of claim 39, wherein:
said parenteral administration is subcutaneous, intramuscular, or intravenous.


SEQUENCE LISTING (74 sequences)

SEQ ID NO: 1 - protein, 4 aa, Homo sapiens
FWQQ

SEQ ID NO: 2 - protein, 5 aa, Homo sapiens
HFWQQ

SEQ ID NO: 3 - protein, 6 aa, Homo sapiens
  PROPEP (1)..(6)
RHFWQQ

SEQ ID NO: 4 - protein, 7 aa, Homo sapiens
  PROPEP (1)..(6)
RHFWQQD

SEQ ID NO: 5 - protein, 8 aa, Homo sapiens
  PROPEP (1)..(6)
RHFWQQDE

SEQ ID NO: 6 - protein, 9 aa, Homo sapiens
  PROPEP (1)..(6)
RHFWQQDEP

SEQ ID NO: 7 - protein, 9 aa, Homo sapiens
  PROPEP (1)..(6)
  VARIANT (9)..(9)
RHFWQQDEH

SEQ ID NO: 8 - protein, 9 aa, Homo sapiens
  PROPEP (1)..(6)
  VARIANT (9)..(9)
RHFWQQDER

SEQ ID NO: 9 - protein, 10 aa, Homo sapiens
  PROPEP (1)..(6)
RHFWQQDEPP

SEQ ID NO: 10 - protein, 10 aa, Homo sapiens
  PROPEP (1)..(6)
  VARIANT (9)..(9)
RHFWQQDEHP

SEQ ID NO: 11 - protein, 10 aa, Homo sapiens
  PROPEP (1)..(6)
  VARIANT (9)..(9)
RHFWQQDERP

SEQ ID NO: 12 - protein, 10 aa, Homo sapiens
  PROPEP (1)..(6)
  VARIANT (10)..(10)
RHFWQQDEPR

SEQ ID NO: 13 - protein, 10 aa, Homo sapiens
  PROPEP (1)..(6)
  VARIANT (9)..(10)
RHFWQQDEHR

SEQ ID NO: 14 - protein, 10 aa, Homo sapiens
  PROPEP (1)..(6)
  VARIANT (9)..(10)
RHFWQQDERR

SEQ ID NO: 15 - protein, 4 aa, Rattus norvegicus
FWQQ

SEQ ID NO: 16 - protein, 5 aa, Rattus norvegicus
EFWQQ

SEQ ID NO: 17 - protein, 6 aa, Rattus norvegicus
  PROPEP (1)..(6)
WEFWQQ

SEQ ID NO: 18 - protein, 7 aa, Rattus norvegicus
  PROPEP (1)..(6)
WEFWQQD

SEQ ID NO: 19 - protein, 8 aa, Rattus norvegicus
  PROPEP (1)..(6)
WEFWQQDE

SEQ ID NO: 20 - protein, 9 aa, Rattus norvegicus
  PROPEP (1)..(6)
WEFWQQDEP

SEQ ID NO: 21 - protein, 10 aa, Rattus norvegicus
  PROPEP (1)..(6)
WEFWQQDEPQ

SEQ ID NO: 22 - protein, 4 aa, Macaca fascicularis
FWQQ

SEQ ID NO: 23 - protein, 5 aa, Macaca fascicularis
HFWQQ

SEQ ID NO: 24 - protein, 6 aa, Macaca fascicularis
  PROPEP (1)..(6)
RHFWQQ

SEQ ID NO: 25 - protein, 7 aa, Macaca fascicularis
  PROPEP (1)..(6)
RHFWQQD

SEQ ID NO: 26 - protein, 8 aa, Macaca fascicularis
  PROPEP (1)..(6)
RHFWQQDE

SEQ ID NO: 27 - protein, 9 aa, Macaca fascicularis
  PROPEP (1)..(6)
RHFWQQDEP

SEQ ID NO: 28 - protein, 10 aa, Macaca fascicularis
  PROPEP (1)..(6)
RHFWQQDEPP

SEQ ID NO: 29 - protein, 4 aa, Sus scrofa
FWQQ

SEQ ID NO: 30 - protein, 5 aa, Sus scrofa
HFWQQ

SEQ ID NO: 31 - protein, 6 aa, Sus scrofa
  PROPEP (1)..(6)
RHFWQQ

SEQ ID NO: 32 - protein, 7 aa, Sus scrofa
  PROPEP (1)..(6)
RHFWQQD

SEQ ID NO: 33 - protein, 8 aa, Sus scrofa
  PROPEP (1)..(6)
RHFWQQDD

SEQ ID NO: 34 - protein, 9 aa, Sus scrofa
  PROPEP (1)..(6)
RHFWQQDDP

SEQ ID NO: 35 - protein, 10 aa, Sus scrofa
  PROPEP (1)..(6)
RHFWQQDDPQ

SEQ ID NO: 36 - protein, 4 aa, Mus musculus
VWQQ

SEQ ID NO: 37 - protein, 5 aa, Mus musculus
HVWQQ

SEQ ID NO: 38 - protein, 6 aa, Mus musculus
  PROPEP (1)..(6)
WHVWQQ

SEQ ID NO: 39 - protein, 7 aa, Mus musculus
  PROPEP (1)..(6)
WHVWQQD

SEQ ID NO: 40 - protein, 8 aa, Mus musculus
  PROPEP (1)..(6)
WHVWQQDE

SEQ ID NO: 41 - protein, 9 aa, Mus musculus
  PROPEP (1)..(6)
WHVWQQDEP

SEQ ID NO: 42 - protein, 10 aa, Mus musculus
  PROPEP (1)..(6)
WHVWQQDEPQ

SEQ ID NO: 43 - protein, 4 aa, Artificial Sequence
  Description of Artificial Sequence Synthetic Peptide
FWQQ

SEQ ID NO: 44 - protein, 4 aa, Artificial Sequence
  Description of Artificial Sequence Synthetic Peptide
VWQQ

SEQ ID NO: 45 - protein, 5 aa, Artificial Sequence
  Description of Artificial Sequence Synthetic Peptide
HFWQQ

SEQ ID NO: 46 - protein, 5 aa, Artificial Sequence
  Description of Artificial Sequence Synthetic Peptide
EFWQQ

SEQ ID NO: 47 - protein, 5 aa, Artificial Sequence
  Description of Artificial Sequence Synthetic Peptide
HVWQQ

SEQ ID NO: 48 - protein, 6 aa, Artificial Sequence
  Description of Artificial Sequence Synthetic Peptide
RHFWQQ

SEQ ID NO: 49 - protein, 6 aa, Artificial Sequence
  Description of Artificial Sequence Synthetic Peptide
WEFWQQ

SEQ ID NO: 50 - protein, 6 aa, Artificial Sequence
  Description of Artificial Sequence Synthetic Peptide
WHVWQQ

SEQ ID NO: 51 - protein, 7 aa, Artificial Sequence
  Description of Artificial Sequence Synthetic Peptide
RHFWQQD

SEQ ID NO: 52 - protein, 7 aa, Artificial Sequence
  Description of Artificial Sequence Synthetic Peptide
WEFWQQD

SEQ ID NO: 53 - protein, 7 aa, Artificial Sequence
  Description of Artificial Sequence Synthetic Peptide
WHVWQQD

SEQ ID NO: 54 - protein, 8 aa, Artificial Sequence
  Description of Artificial Sequence Synthetic Peptide
RHFWQQDE

SEQ ID NO: 55 - protein, 8 aa, Artificial Sequence
  Description of Artificial Sequence Synthetic Peptide
WEFWQQDE

SEQ ID NO: 56 - protein, 8 aa, Artificial Sequence
  Description of Artificial Sequence Synthetic Peptide
WHVWQQDE

SEQ ID NO: 57 - protein, 9 aa, Artificial Sequence
  Description of Artificial Sequence Synthetic Peptide
RHFWQQDEP

SEQ ID NO: 58 - protein, 9 aa, Artificial Sequence
  Description of Artificial Sequence Synthetic Peptide
RHFWQQDEH

SEQ ID NO: 59 - protein, 9 aa, Artificial Sequence
  Description of Artificial Sequence Synthetic Peptide
RHFWQQDER

SEQ ID NO: 60 - protein, 9 aa, Artificial Sequence
  Description of Artificial Sequence Synthetic Peptide
WEFWQQDEP

SEQ ID NO: 61 - protein, 9 aa, Artificial Sequence
  Description of Artificial Sequence Synthetic Peptide
WHVWQQDEP

SEQ ID NO: 62 - protein, 10 aa, Artificial Sequence
  Description of Artificial Sequence Synthetic Peptide
RHFWQQDEPP

SEQ ID NO: 63 - protein, 10 aa, Artificial Sequence
  Description of Artificial Sequence Synthetic Peptide
RHFWQQDEHP

SEQ ID NO: 64 - protein, 10 aa, Artificial Sequence
  Description of Artificial Sequence Synthetic Peptide
RHFWQQDERP

SEQ ID NO: 65 - protein, 10 aa, Artificial Sequence
  Description of Artificial Sequence Synthetic Peptide
RHFWQQDEPR

SEQ ID NO: 66 - protein, 10 aa, Artificial Sequence
  Description of Artificial Sequence Synthetic Peptide
RHFWQQDEHR

SEQ ID NO: 67 - protein, 10 aa, Artificial Sequence
  Description of Artificial Sequence Synthetic Peptide
RHFWQQDERR

SEQ ID NO: 68 - protein, 10 aa, Artificial Sequence
  Description of Artificial Sequence Synthetic Peptide
WEFWQQDEPQ

SEQ ID NO: 69 - protein, 10 aa, Artificial Sequence
  Description of Artificial Sequence Synthetic Peptide
WHVWQQDEPQ

SEQ ID NO: 70 - DNA, 30 bp, Homo sapiens
cggcatttctggcagcaagatgaacccccc

SEQ ID NO: 71 - DNA, 30 bp, Rattus norvegicus
tgggaattctggcagcaagatgaaccccag

SEQ ID NO: 72 - DNA, 30 bp, Macaca fascicularis; identical to SEQ ID NO: 70

SEQ ID NO: 73 - DNA, 30 bp, Sus Scrofa
cggcatttctggcagcaagatgatccccag

SEQ ID NO: 74 - DNA, 30 bp, Mus musculus
tggcatgtttggcagcaagatgaaccccag